(19) United States
(12) Patent Application Publication
(10) Pub. No.: US 2025/0382685 A1
(43) Pub. Date: Dec. 18, 2025

(54) METHOD OF PRODUCING A MICROALLOYED MAGNESIUM ALLOY

(71) Applicant: MgSANA Corp., Mobile, AL (US)

(72) Inventor: Amir ELIEZER, Omer (IL)

(21) Appl. No.: 19/235,028

(22) Filed: Jun. 11, 2025

(30) Foreign Application Priority Data
Jun. 14, 2024 (DE) 10 2024 116 789.0

(51) Int. Cl. C22C 23/04

(52) U.S. Cl. CPC C22C 23/04

(57) ABSTRACT

The disclosure relates to a method of producing a microalloyed magnesium alloy. The magnesium alloy may include 1-2 weight % zinc (Zn), 0.5-1 weight % manganese (Mn), and 0.2-0.8 weight % calcium (Ca). A billet may be heated to a temperature between 220 to 340 degrees Celsius (° C.), and the billet may be drawn to a rod of the microalloyed magnesium alloy in the heated state.

### CROSS REFERENCE

The present Application for Patent claims the benefit of German Patent Application No. 102024116789.0 by Amir Eliezer entitled “Method of Producing a Microalloyed Magnesium Alloy,” filed Jun. 14, 2024, which is assigned to the assignee hereof and is incorporated herein by reference in its entirety.

### TECHNICAL FIELD

The present disclosure relates to a microalloyed magnesium alloy and a method for production thereof. In particular, the present disclosure relates to a bioresorbable magnesium alloy for medical applications.

### BACKGROUND

Document WO2019/002277A1 shows a bioresorbable magnesium alloy, which comprises calcium, manganese, and zinc.

### SUMMARY

It is an object of the present disclosure to provide a magnesium alloy, in particular for medical applications, with improved mechanical properties.

The object of the present disclosure is achieved by a method for producing a microalloyed magnesium alloy.

Additional examples of the present disclosure are subject of the dependent claims, the description, and the drawings.

The present disclosure relates to method of producing a microalloyed magnesium alloy. The magnesium alloy comprises 1-2 weight % zinc (Zn) (e.g., the alloy comprises between 1% and 2% Zn by weight), 0.5-1 weight % manganese (Mn) (e.g., the alloy comprises between 0.5% and 1% Mn by weight), and 0.2-0.8 weight % calcium (Ca) (e.g., the alloy comprises between 0.2% and 0.8% Ca by weight). In some examples, the alloy comprises 97 weight % magnesium (Mg), except of Mn, Ca, and Mg, not any other alloying elements. The magnesium alloy is free of any rare earth elements.

According to the present disclosure, a billet is heated to a temperature between 220 to 340 degrees Celsius (° C.) and then drawn to a rod in the heated state. The drawing process can be performed by using an extrusion machine.

According to an example, the billet is heated to a temperature of 400 to 500° C.

In some examples, then the billed is cooled down, preferably to room temperature.

In some examples, then the billet to the temperature of 300 to 400° C. and drawn to the rod in the heated state of 220 to 340° C.

In some examples, the billet is drawn with a temperature of 240 to 310° C.

In some examples, the billet is heated to a temperature of 400 to 500° C. for 2 to 8 hours.

According to an example, the diameter of the billet is reduced by drawing the billet in two steps. This results in reducing the grain size to a preferred range. In particular, a grain size of 0.7 to 2.4 micrometers (μm) result is an improved mechanical strength and in an increased corrosion resistance also.

According to an example, the diameter of the billet is reduced in a first drawing step to a 0.1 to 0.5 smaller diameter. The diameter of the billet can be reduced in a second drawing step to a 0.1 to 0.3 smaller diameter.

At least the second drawing step is performed at the temperature of 220 to 340° C., preferably at 240 to 310° C.

In further the present disclosure relates to a microalloyed magnesium alloy, in particular being produced with a method as described above.

The magnesium alloy comprises 1-2 weight % Zn, 0.5-1 weight % Mn and 0.2-0.8 weight % Ca.

The magnesium alloy has grain size between 0.7 and 2.4 μm, a tensile yield strength (TYS) of more than 200 megapascals (MPa) and an elongation at fracture of more than 18%.

In some examples, the magnesium alloy has a TYS of 250-380 MPa.

In some examples, the magnesium alloy has a TYS of more than 300 MPa.

In some examples, the magnesium alloy has an elongation at fracture of 18 to 35%.

In some examples, the magnesium alloy has an ultimate tensile strength of more than 250 MPa, in particular of 250 MPa to 390 MPa.

Elongation at fracture (A), TYS and ultimate tensile strength (UTS) are determined according to DIN EN ISO 6892-1-2020-06. A material test system (MTS) universal testing machine 810 with tactile strain gauge can be used.

The present disclosure further relates to a medical implant which comprises, in particular which consists of the microalloyed magnesium alloy.

The alloy is biodegradable. According to an example, the alloy comprises a coating, particularly a calcium phosphate coating.

### BRIEF DESCRIPTION OF THE DRAWINGS

FIGS. 1 and 2 are microscopic images of a magnesium alloy according to an example of the present disclosure.

FIG. 3 is a flow chart of a process for producing an implant according to an example of the present disclosure.

FIG. 4 and FIG. 5 are Nyquist plots of alloys which had been drawn at different temperatures.

### DETAILED DESCRIPTION

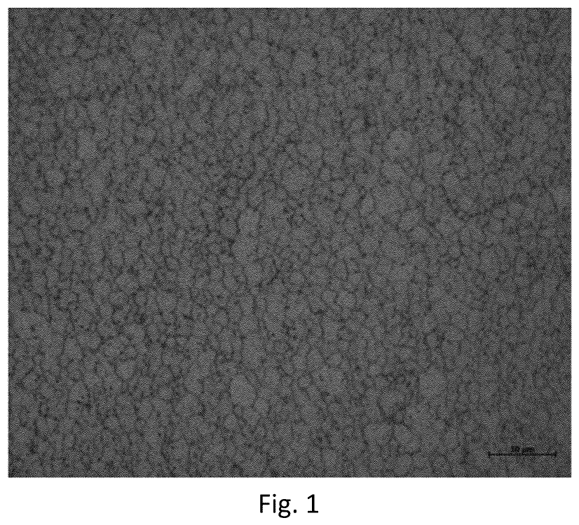

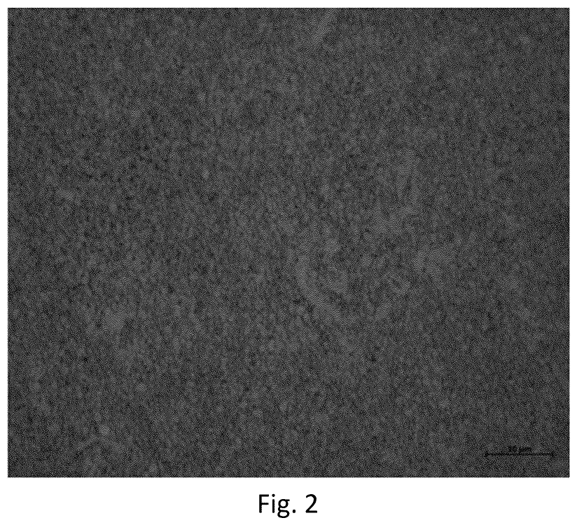

FIGS. 1 and 2 are microscopic images of a magnesium alloy according to an example of the present disclosure.

For the alloy according to FIG. 1, the billet had been drawn at a temperature of 310° C. The drawn alloy has a TYS of approximately 250 MPa, an UTS of approximately 290 MPa, and an elongation A of approximately 25%. The grain size is 1.7 μm.

For the alloy according to FIG. 2, the billet had been drawn at a temperature of 240° C. The drawn alloy has a TYS of approximately 360 MPa, an UTS of approximately 350 MPa, and an elongation A of approximately 15%. The grain size is 1.0 μm.

Hence, a higher temperature results in slightly reduced mechanical strength but results in a higher elongation.

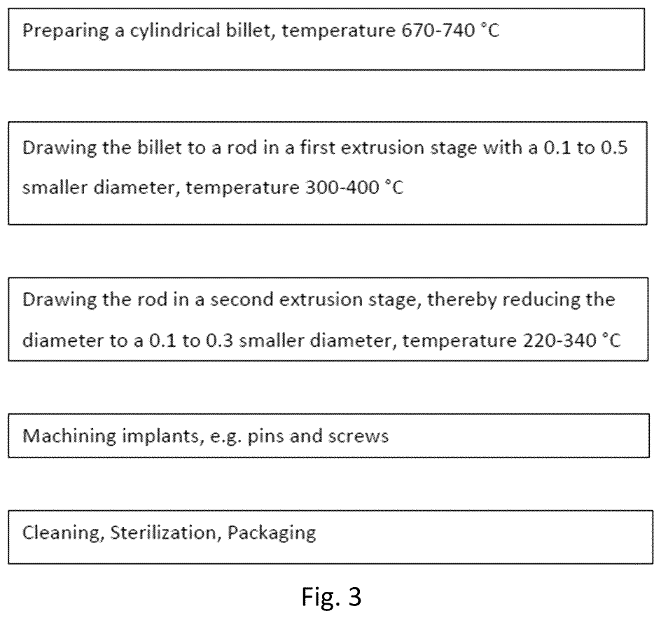

FIG. 3 shows a process for producing an implant according to an example of the present disclosure.

First, a cylindrical billet is casted, where the billet temperature is between 670 to 740° C.

Then, the billet is drawn to a rod of the microalloyed magnesium alloy in a first extrusion stage with a 0.1 to 0.5 smaller diameter (e.g., 10% to 50% smaller) as the billet, where the temperature is 300-400° C.

Then, the rod is drawn in a second extrusion stage, thereby reducing the diameter to a 0.1 to 0.3 smaller diameter (e.g., 10% to 30% smaller), where the temperature is 220-340° C.

Then, the rod is machined to an implant (e.g. to a pin or screw).

Finally, the implant is cleaned, sterilized, and packaged.

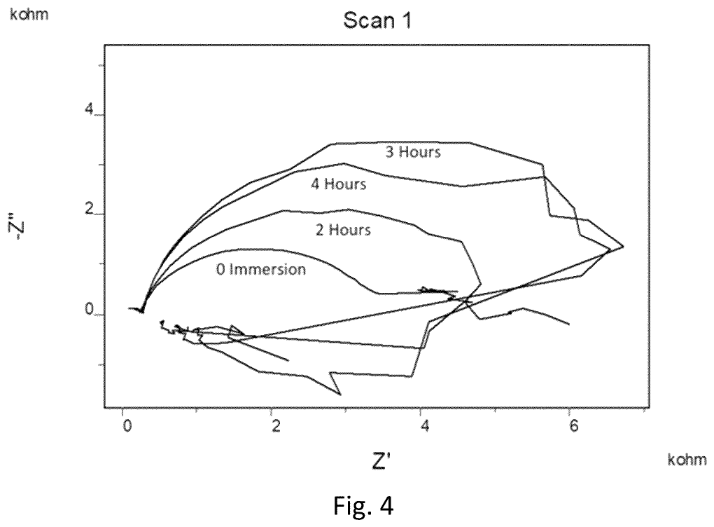

FIG. 4 is a Nyquist plot of an Mg alloy, which had been drawn at 240° C., and which has a grain size of 0.9 μm. The alloy had been immersed in a saline solution for 0, 2, 3 and 4 hours.

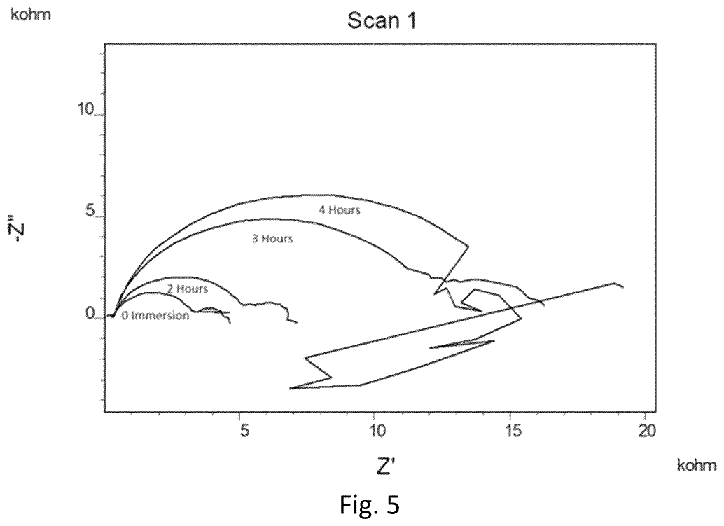

FIG. 5 is a Nyquist plot of an Mg alloy, which had been drawn at 310° C., and which has a grain size of 1.5 μm.

The Nyquist impedance can used to determine the corrosion resistance of the alloy.

Regarding the impedance, the alloy according to FIG. 4 has a high corrosion resistance and has a high mechanical strength.

Overall, at 0, 1, 3, 4 hours of exposure the vertical Nyquist axis is up to 4 Z″ kiloohm (kOhm), and the horizontal axis is up to 8 Z′ kOhm.

The alloy according to FIG. 5 has at 0, 1, 3, 4 hours of exposure, a vertical Nyquist axis up to 7 Z″ kOhm and a horizontal up to 18 Z′ kOhm.

The Nyquist plot shows the relationship between grain size, drawing temperature, and corrosion resistance.

In comparison to FIG. 4, the alloy according to FIG. 5 was drawn with a higher process temperature, which results in a larger grain size, which results in a higher impedance, and which shows a higher corrosion resistance.

The lower process temperature results in a smaller grain size and improved mechanical properties. A smaller grain size results, due to Gibbs Energy and higher amount of grain boundaries, in a smaller corrosion resistance.

What is claimed is:

1. A method of producing a microalloyed magnesium alloy, the method comprising:
heating a billet to a temperature between 220 to 340 degrees Celsius (° C.); and
drawing the billet to a rod of the microalloyed magnesium alloy in a heated state, wherein the microalloyed magnesium alloy comprises 1-2 weight % zinc (Zn), 0.5-1 weight % manganese (Mn), and 0.2-0.8 weight % calcium (Ca).

2. The method of claim 1, further comprising:
heating the billet to a temperature between 400 to 500° C.;
cooling down the heated billet;
heating the billet to the temperature between 240 to 340° C.;
drawing the billet to the rod in the heated state between 220 to 340° C.

3. The method of claim 1, wherein the billet is drawn with a temperature between 240 to 310° C.

4. The method of claim 1, wherein the billet is heated to a temperature between 400 to 600° C. for 2 to 8 hours.

5. The method of claim 1, wherein a diameter of the billet is reduced by drawing the billet in two steps.

6. The method of claim 5, wherein the diameter of the billet is reduced in a first drawing step to a 0.1 to 0.5 smaller diameter.

7. The method of claim 5, wherein the diameter of the billet is reduced in a second drawing step to a 0.1 to 0.3 smaller diameter.

8. A microalloyed magnesium alloy, comprising:
1-2 weight % zinc (Zn);
0.5-1 weight % manganese (Mn); and
0.2-0.8 weight % calcium (Ca), wherein the microalloyed magnesium alloy has grain size between 0.7 and 2.4 micrometers (μm), a tensile yield strength (TYS) of more than 200 megapascals (MPa), and an elongation at fracture of more than 10%.

9. The microalloyed magnesium alloy of claim 8, wherein the microalloyed magnesium alloy has an elongation at fracture of 18%.

10. The microalloyed magnesium alloy of claim 8, wherein the microalloyed magnesium alloy has a TYS of 250-390 MPa.

11. The microalloyed magnesium alloy of claim 8, wherein the microalloyed magnesium alloy has a TYS of more than 300 MPa.

12. The microalloyed magnesium alloy of claim 8, wherein the microalloyed magnesium alloy has an elongation at fracture of 18 to 35%.

13. The microalloyed magnesium alloy of claim 8, wherein the microalloyed magnesium alloy has an ultimate tensile strength of more than 250 MPa.

14. The microalloyed magnesium alloy of claim 8, wherein the microalloyed magnesium alloy has an ultimate tensile strength of 250 MPa to 390 MPa.

15. A medical implant, comprising:
a microalloyed magnesium alloy, wherein the microalloyed magnesium alloy comprises 1-2 weight % zinc (Zn), 0.5-1 weight % manganese (Mn), and 0.2-0.8 weight % calcium (Ca), wherein the microalloyed magnesium alloy has grain size between 0.7 and 2.4 micrometers (μm), a tensile yield strength (TYS) of more than 200 megapascals (MPa), and an elongation at fracture of more than 10%.

16. The medical implant of claim 15, wherein the microalloyed magnesium alloy has an elongation at fracture of 18%.

17. The medical implant of claim 15, wherein the microalloyed magnesium alloy has a TYS of 250-390 MPa.

18. The medical implant of claim 15, wherein the microalloyed magnesium alloy has a TYS of more than 300 MPa.

19. The medical implant of claim 15, wherein the microalloyed magnesium alloy has an elongation at fracture of 18 to 35%.

20. The medical implant of claim 15, wherein the microalloyed magnesium alloy has an ultimate tensile strength of more than 250 MPa.
